(19) United States
(12) Patent Application Publication
(10) Pub. No.: US 2007/0277831 A1
(43) Pub. Date: Dec. 6, 2007

(54) Nasal assembly and method of positioning nasogastric tubing

(76) Inventor: Reba Luhrs, Kirkwood, MO (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 11/444,707

(22) Filed: Jun. 1, 2006

(51) Int. Cl. A61M 29/00; A62B 7/00; A61M 15/08

(52) U.S. Cl. 128/207.18; 128/200.24; 606/196

(57) ABSTRACT

A nasal assembly for positioning nasogastric tubing within a patient's nose. The nasal assembly comprises a nasal adapter having a lumen disposed there through for guiding the nasogastric tubing within the nasal adapter. The nasal adapter further has a flange and a tubular portion extending from the flange. The assembly further comprises a sleeve that removeably surrounds a portion of the tubular portion. The sleeve has a body comprising of a compressible material. The body has a front side, a back side and a bore disposed there between for guiding the nasogastric tubing within the sleeve and beyond the back side. The front side contacts against the flange and the back side contacts against the patient's nose wherein the back side remains free from entering the patient's nose while positioning the nasogastric tubing within the patient's nose.

### CROSS-REFERENCE TO RELATED APPLICATIONS

Not Applicable.

### STATEMENT REGARDING FEDERALLY SPONSORED RESEARCH

Not Applicable.

### BACKGROUND OF THE DISCLOSURE

The present disclosure relates to a nasal assembly, and in particular, it relates to a sleeve that positions nasogastric tubing within a patient's nose while the sleeve remains free from entering the patient's nose and contacts the exterior of the nose in order to protect the skin of the nose.

Tubing inserted through the nasal passage of medical patients introduces oxygen, or air, or other fluid (liquid or gas) treatments into the nasopharyngeal area and directly into the airway or stomach. In addition, tubing inserted through the nasal passage of patients provides a means of introducing fluids into, or extracting fluids from, the gastrointestinal tract.

Once the tubing is inserted through the nasal passage and appropriately positioned to achieve the desired treatment, health care personnel currently use an attachment device to position an insert within the nose in order to secure the nasal tubing within the nose. Treatments administered to the patient by the nasal tube method can require that the tubing remain in place for extended periods of time such as several hours or days. For tubing placement, the inserted tubing requires a particular position for these extended periods for effective treatment. Furthermore, to maintain appropriate health care standards and to minimize trauma and irritation to the patient, the attachment device and insert require periodic replacement while the nasal tubing remains in place. As such, the tubing must be attached to, and easily detachable from, the insert to effectively deliver fluids to or remove fluids from the patient. Second, the insert must be comfortably secured to, and easily detachable from, the patient. Convenience and time efficiency regarding attachment are important considerations for the health care personnel and the patient.

A common method currently used to attach nasal tubing to patients involves taping the tube to the patient's face. Generally, surgical tape or similarly employed adhesive pads are used to anchor the nasal tubing to the skin of the patient. The patient's nose, however, can become irritated and sore as a result of repeated application and removal of the tape. Another common method involves inserting a portion of the insert inside the nose while attaching another portion of the insert to the outside of the nose. In particular, this method attaches the outside portion of the insert to the alar wing of the nose. The insertion of the insert into the nose causes irritation of the nasal membranes leading to mucous blockage. Furthermore, the attachment of the insert to alar wing of the nose by the attachment device leads to skin erosion on the nose. This skin erosion leads to painful infections and to prolonged post treatment care. Accordingly, health care personnel and patients require positioning of nasal tubing without irritating the skin on the nose.

### BRIEF SUMMARY OF THE DISCLOSURE

The present disclosure relates to a nasal assembly that does not enter the nose while it positions nasogastric tubing within the nose. The nasal assembly comprises a nasal adapter having a lumen for guiding the nasogastric tubing within the nasal adapter as the tubing is inserted into the airway or stomach. The nasal adapter further has a flange and a tubular portion extending from the flange. The assembly further comprises a sleeve that removeably surrounds a portion of the tubular portion. The sleeve has a body comprising of a compressible material. The body has a front side, a back side and a bore disposed there between positioning the lumen within the sleeve. The front side abuts against the flange and the back side abuts against the patient's nose wherein the back side remains free from entering the patient's nose while positioning the nasogastric tubing within the patient's nose.

The present disclosure also relates to a method of positioning the sleeve in communication with a patient's nose for inserting nasogastric tubing within the patient's nose. The method comprises removeably positioning the sleeve around a tubular portion of the nasal adapter. The front of the sleeve is then moved against the flange of the nasal adapter. Next, the method comprises inserting the nasogastric tubing within the lumen of the nasal adapter, wherein the nasogastric tubing extends beyond the sleeve. Then, the method comprises inserting the nasogastric tubing that extends beyond the sleeve into the patient's nose. The back side of the sleeve is positioned against the alar wing of the patient's nose wherein the back side of the sleeve remains free from entering the patient's nose while the nasogastric tubing is positioned within the patient's nose.

The foregoing and other objects, features, and advantages of the disclosure as well as presently preferred embodiments thereof will become more apparent from the reading of the following description in connection with the accompanying drawings.

### BRIEF DESCRIPTION OF THE SEVERAL VIEWS OF THE DRAWINGS

In the accompanying drawings which form part of the specification:

FIG. 1 is a perspective view illustrating a nasal assembly which comprises a nasal adapter and sleeve constructed in accordance with and embodying the present disclosure;

FIG. 2 is a side view of the nasal adapter and sleeve of FIG. 1 and further illustrating a medicinal layer positioned against the sleeve in accordance with the present disclosure;

FIG. 3 is a side elevational view of the nasal assembly positioned against a patient's nose wherein the sleeve remains free from entering the nose;

FIG. 4 is a side view of another embodiment of the nasal assembly illustrating perforations disposed through the nasal adapter and sleeve; and

FIG. 5 is a perspective view of another embodiment of the sleeve illustrating tapered surfaces of the nasal adapter and sleeve; and

FIG. 6 is a flowchart illustrating step of positioning the nasogastric tube within the patient's nose.

Corresponding reference numerals indicate corresponding parts throughout the several figures of the drawings.

### DESCRIPTION OF THE PREFERRED EMBODIMENT

The following detailed description illustrates the disclosure by way of example and not by way of limitation. The description clearly enables one skilled in the art to make and use the disclosure, describes several embodiments, adaptations, variations, alternatives, and uses of the disclosure, including what is presently believed to be the best mode of carrying out the disclosure.

Referring to the drawings, a nasal assembly A (FIG. 1) is shown. The nasal assembly A comprises a nasal adapter 10, a sleeve 12 and a medicinal layer 14. The nasal assembly A positions nasogastric tubing 15 within a patient's nose 16 (FIG. 3) without substantially irritating or eroding the skin of the nose 16. In one embodiment, the nasogastric tubing 15 comprises a soft rubber like material.

As shown in FIG. 1, the nasal adapter 10 comprises an adapter body 18 having a distal end 20, a proximal end 22, an inner wall 24 and an outer wall 26. The inner wall 24 and the outer wall 26 define a lumen 28 through the inner wall 24. The lumen 28 guides the nasogastric tubing 15 within the nasal adapter 10.

At the distal end 20, a flange 30 extends outward from the lumen 28. In one embodiment, the flange 30 comprises a substantially straight flange 30. Further as shown, at the proximal end 22, a tubular portion 32 extends from the flange 30. The flange 30 is configured to direct the nasogastric tubing 15 into the lumen 28 while the tubular portion 32 is configured to direct the nasogastric tubing 15 out of the nasal adapter 10. A slight taper may exist at the intersection of the flange 30 and the tubular portion 32. Additionally, the flange 30 is configured to assist the health care personnel in handling the nasal adapter 10 since the personnel can easily grasp the flange 30.

As shown in FIG. 1, the sleeve 12 removably surrounds a portion of the tubular portion 32. The sleeve 12 includes a sleeve body 34 comprising of a compressible material such as, but not limited to, a foam material, a gel material or a rubber material. The sleeve body 34 has a front side 36, a back side 38 and a bore 40 disposed there through. The sleeve body 34 also includes an outer surface 42. The bore 40 is configured to further guide the nasogastric tubing 15 within the sleeve 12 and beyond the back side 38 of the sleeve 12. As shown, the front side 36 of the sleeve body 34 contacts against the flange 30, wherein the outer surface 42 of the sleeve body extends beyond the flange 30. In another embodiment, the outer surface 42 matches the dimensions of the flange 30.

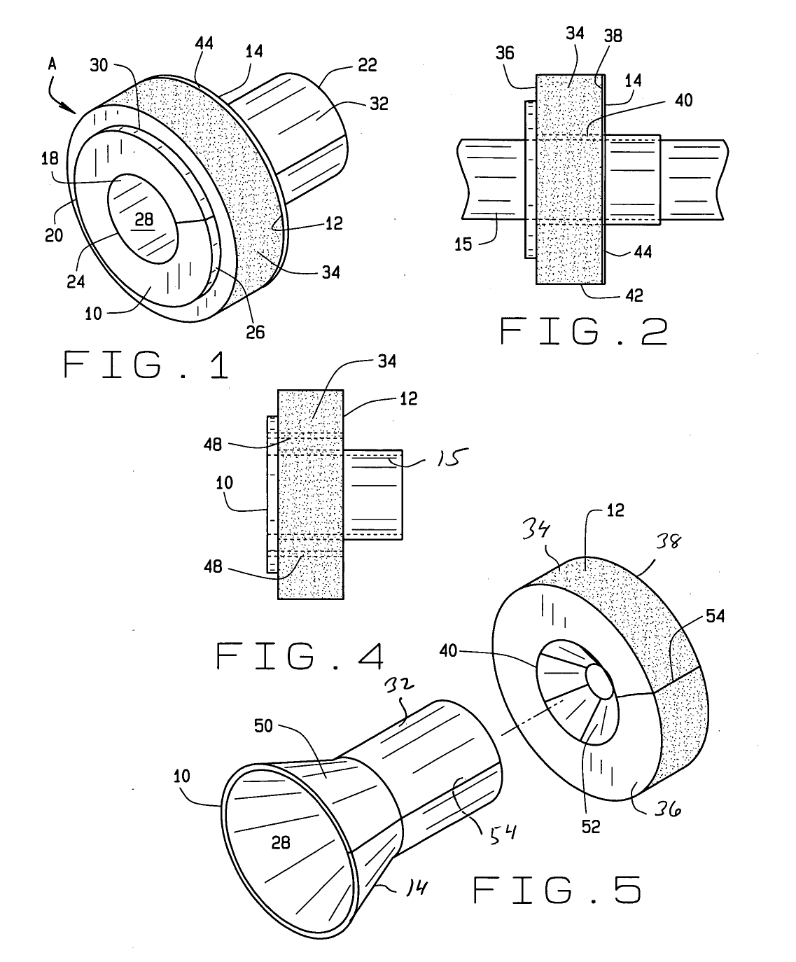

As illustrated in FIG. 2, the medicinal layer 14 contacts the back side 38 of the sleeve body 34. The medicinal layer 14 also comprises, in one embodiment, a compressible material. The medicinal layer 14 preferably includes a local anesthetic 44 such as lidocaine disbursed throughout. In one embodiment, the medicinal layer 14 comprises the local anesthetic 44. In other words, the local anesthetic 44 is directly applied to the back side 38 of the sleeve body 34. In another embodiment, the medicinal layer 14 comprises an anti-microbial material, such as a silver material that inhibits growth of bacteria or viruses. As shown in FIG. 2, the lumen 28 of the nasal adapter 10 aligns with the bore 40 of the sleeve 12 to position the nasogastric tubing 15 through the nasal adapter 10 and beyond the sleeve 12. In one embodiment, an adhesive may attach together the sleeve 12 and the nasal adapter 10.

Referring to FIG. 3, the nasal gastric assembly A is shown positioning the nasogastric tubing 15 within the patient's nose 16. The front side 36 of the sleeve body 34 contacts a side of the flange 30 while the back side 38 contacts an outside portion of the patient's nostril. Since the outer surface 42 of the sleeve 12 extends beyond the flange 30, the back side 38 of the sleeve body remains free from entering the patient's nose 16 while the nasal assembly A positions the nasogastric tubing 15 within the patient's nose 16. In particular, the back side 38 contacts an alar wing 46 of the nose 16. The alar wing 46 may comprise the apex, the exterior nostrils and nares of the nose 16. Since the sleeve 12 comprises a compressible material, the back side 38 of the sleeve contacting the alar wing 46 does not substantially irritate and/or erode the skin on the outside of the nose 16. Further, since the sleeve 12 does not enter the nose 16, the sleeve 12 does not irritate the mucus membranes.

Turning to FIG. 4, another embodiment of the nasal assembly A is shown. In this embodiment, one or more perforations 48 are disposed through the nasal adapter 10 and sleeve 12. These perforations 48 are sized and shaped to allow air to travel between the environment and the patient's nose 16. The perforations 48 are configured as having a sufficient cross sectional area to permit the patient to breathe. In other words, when the nasal adapter 10 and sleeve 12 position the nasogastric tubing 15 within the patient's nose 16, the patient can still breathe through the nasal gastric assembly A via the perforations 48.

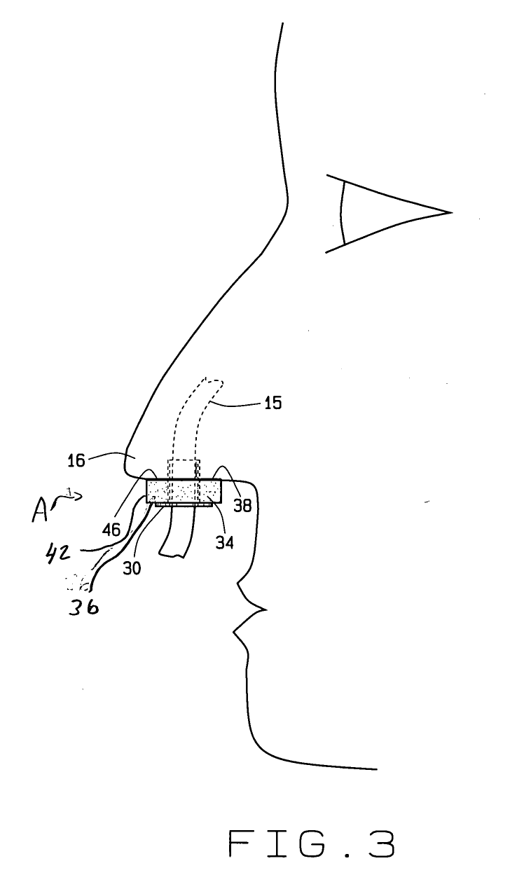

Turning to FIG. 5, another embodiment of the nasal adapter 10 and sleeve 12 are shown. In this embodiment, the nasal adapter 10 includes a tapered or curved outer wall 50 and a more pronounced entrance for the lumen 28. Accordingly, the surface of the sleeve body 34 matches the configuration of the nasal adapter 10. As such, the front side 36 of the sleeve 12 also includes a more tapered configuration to present a more pronounced entrance to the bore 40 of the sleeve 12. Further, as shown, in this embodiment the sleeve 12 may include a slit 54 that extends along the length of the sleeve between the front side 36 and the back side 38. The slit 54 allows the sleeve to be insertable around the nasogastric tubing 15 (FIGS. 1-4) and tubular portion 32 of the nasal adapter 10. The slit 54 allows the sleeve to be opened to fit around the tubing 15. In one embodiment, the adapter 14 may also include a slit so that the adapter may be opened to fit around the tubing 15.

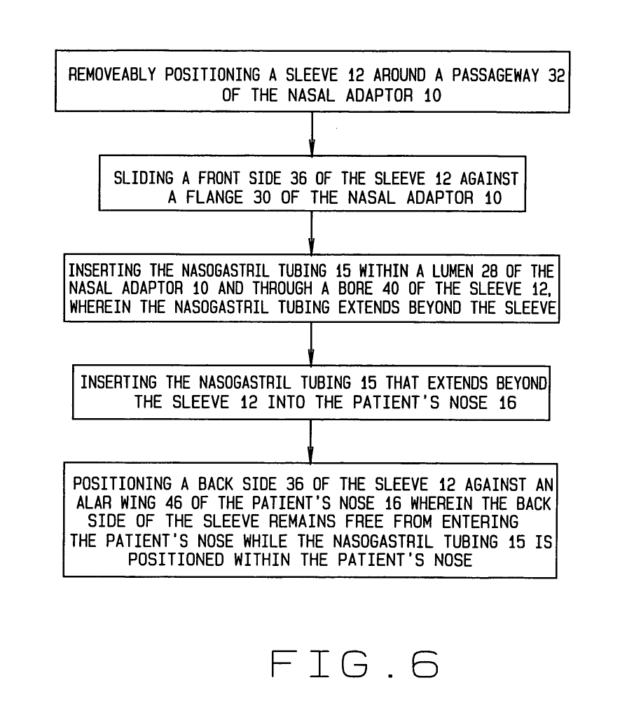

Turning to the flowchart of FIG. 6 and referring to FIGS. 1-5, the nasal sleeve 12 of the present disclosure assists in positioning the nasogastric tubing 15 within the patient's nose 16 without irritating or eroding the skin of the patient's nose 16. During insertion of the nasal tubing 15, the healthcare personnel positions the patient on the side of the bed or chair and measures the nasogastric tubing 15 from the tip of the nose to the earlobe and down to the breast bone. The healthcare personnel then marks this point of the nasogastric tubing 15 wherein this length will allow the tube to properly insert within the gastrinal/intestinal track and into the stomach. The healthcare personnel inserts an end of the nasogastric tubing 15 within the lumen 28 of the nasal adapter 10. The healthcare personnel then removably positions the sleeve 12 around the tubular portion 32 of the nasal adapter 10. The healthcare personnel may slide the sleeve 12 along an end of the nasogastric tubing 15 and around the tubular portion 32 to contact the sleeve with the nasal adapter 10. The healthcare personnel may also insert the tubular portion 32 of the nasal adapter 10 through the slit 54 of the sleeve 12.

In removably positioning the sleeve 12 around the tubular portion 32 of the nasal adapter 10, the healthcare personnel slides the front side 36 of the sleeve 12 against the flange 30 of the nasal adapter 10. In this position, the personnel may insert the nasogastric tubing 15 through the lumen 28 and beyond the sleeve 12. Once the nasogastric tubing 15 extends beyond the sleeve and with the proper length of the nasogastric tubing 15, the personnel lubricates the nasogastric tubing 15 and inserts the tubing 15 through the nostril until it reaches the marked point. The patient may take sips of water to assist passing the tube into the gastric and intestinal track. A portion of the tubular portion 32 that is not covered by the sleeve 12 may also insert within the patient's nose 16. In doing so, the healthcare personnel positions the back side 38 of the sleeve 12 against the alar wing 46 of the patient's nose 16 where in this back side 38 remains free from entering the patient's nose 16 while the nasogastric tubing 15 is positioned within the patient's nose 16. Since the sleeve body 34 extends beyond the flange 30, the sleeve body 34 does not enter the patient's nose 16. Furthermore, since the sleeve body 34 comprises a compressible material, the sleeve body 34 prevents irritations and/or erosion of the skin of the alar wing 46 that contacts the sleeve body 34.

To check the location of the nasogastric tubing 15, the personnel health care places a stethoscope over the patient's stomach and with a syringe quickly inserts 3 to 5 cc's of air into the tube while listening with a stethoscope for a “pop”. The pop represents air rushing into the stomach to signal proper insertion of the nasogastric tubing 15.

In an alternative use, the healthcare personnel may apply local anesthetic 44 to the back side 38 of the sleeve prior to positioning the sleeve against the alar wing 46 of the patient's nose 16. Or, the healthcare personnel may apply the medicinal layer 14 to the back side 38 of the sleeve wherein the medicinal layer 14 comprises the local anesthetic 44 distributed throughout this layer 14. In either way, the local anesthetic 44 further reduces discomfort to the patient.

In view of the above, it will be seen that the several objects of the disclosure are achieved and other advantageous results are obtained. As various changes could be made in the above constructions without departing from the scope of the disclosure, it is intended that all matter contained in the above description or shown in the accompanying drawings shall be interpreted as illustrative and not in a limiting sense.

1. A nasal assembly for positioning nasogastric tubing within a patient's nose, comprising:
a nasal adapter having a lumen disposed there through for guiding the nasogastric tubing within the nasal adapter, the nasal adapter further having a flange and a tubular portion extending from the flange; and
a sleeve that removeably surrounds a portion of the tubular portion, the sleeve includes a body comprising of a compressible material, the body having a front side, a back side and a bore disposed there through for guiding the nasogastric tubing within the sleeve and beyond the back side of the sleeve, the front side of the sleeve body contacts against the flange and the back side contacts against the patient's nose wherein the back side remains free from entering the patient's nose while positioning nasogastric tubing within the patient's nose.

2. The nasal assembly of claim 1 wherein the body includes an outer surface that extends beyond the flange.

3. The nasal assembly of claim 1 wherein the front side contacts against a side of the flange and the back side contacts against an alar wing of the nose.

4. The nasal assembly of claim 1 wherein the compressible material comprises a foam material.

5. The nasal assembly of claim 1 wherein the compressible material comprises a rubber material.

6. The nasal assembly of claim 1 further comprising a plurality of perforations disposed through the nasal adapter and the sleeve.

7. The nasal assembly of claim 1 further compromising a medicinal layer positioned on the back side of the body.

8. The nasal assembly of claim 7 wherein the medicinal layer includes a local anesthetic dispersed throughout.

9. The nasal assembly of claim 7 wherein the medicinal layer includes an anti-microbial material dispersed throughout.

10. A nasal sleeve for positioning a nasal adapter which is used to insert nasogastric tubing within a patient's nose, comprising:
a sleeve that removeably surrounds a tubular portion of the nasal adapter, the sleeve having a body comprising of a foam material, the body having a front side, a back side and a bore disposed there between for guiding the nasogastric tubing within the sleeve and beyond the back side, the front side contacts against the flange and the back side contacts against an alar wing of the patient's nose wherein the back side remains free from entering the patient's nose while positioning the nasal adapter in communication with the patient's nose.

11. The nasal sleeve of claim 10 further compromising a medicinal layer positioned on the back side of the body wherein the medicinal layer includes a local anesthetic dispersed throughout.

12. The nasal sleeve of claim 10 further comprising a medicinal layer positioned on the back side of the body wherein the medicinal layer has an anti-microbial material dispersed throughout

13. The nasal sleeve of claim 10 wherein the body further includes a slit that extends from the front side to the back side.

14. A method of positioning a nasal adapter in communication with a patient's nose for inserting nasogastric tubing within the patient's nose, comprising:
removeably positioning a sleeve around a tubular portion of the nasal adapter;
sliding a front side of the sleeve against a flange of the nasal adapter;
inserting the nasogastric tubing within a lumen of the nasal adapter and through a bore of the sleeve, the nasal tubing extending beyond the sleeve;
inserting the nasogastric tubing that extends beyond the sleeve into the patient's nose; and
positioning a back side of the sleeve against the patient's nose wherein the back side of the sleeve remains free from entering the patient's nose while the nasogastric tubing is positioned within the patient's nose.

15. The method of claim 14 wherein removeably positioning the sleeve around the tubular portion of the nasal adapter comprises sliding the sleeve around the tubular portion.

16. The method of claim 14 wherein positioning the back side of the sleeve against the patient's nose comprises positioning the back side in contact with an alar wing of the patient's nose.

17. The method of claim 14 further comprises applying a local anesthetic to the back side of the sleeve prior to positioning the sleeve against the alar wing of the patient's nose.

18. The method of claim 14 further comprising applying a medicinal layer to the back side of the sleeve.

19. The method of claim 14 wherein removeably positioning the sleeve around the tubular portion of the nasal adapter comprises inserting the tubular portion within a slit of the sleeve.

20. The method of claim 14 further comprising applying a local anesthetic to the back side of the sleeve.
